(19) United States
(12) Patent Application Publication
(10) Pub. No.: US 2011/0077430 A1
(43) Pub. Date: Mar. 31, 2011

(54) Method and precursor for production of no-carrier-added N-(4-[18F] fluorobutyl)-Ethacrynic amide

(76) Inventors: Chung-Shan Yu, Taoyuan County (TW); Li-Wu Chiang, Taoyuan County (TW); Hao-Lien Huang, Taoyuan County (TW); Yu-Hsuan Ku, Taoyuan County (TW); Chia-Jung Chen, Taoyuan County (TW); Shao Wei Chen, Taoyuan County (TW); Yean-Hung Tu, Taoyuan County (TW); Mao-Hsung Chang, Taoyuan County (TW); Jenn-Tzong Chen, Taoyuan County (TW); Wuu-Jyn Lin, Taoyuan County (TW)

(21) Appl. No.: 12/569,910

(22) Filed: Sep. 30, 2009

(51) Int. Cl. C07C 233/17

(52) U.S. Cl. 564/169

(57) ABSTRACT

The present invention is related to a precursor for no-carrier-added fluorine-18 labeled ethacrynic acid, N-(4-[¹⁸F]fluorobutyl)-Ethacrynic amide([¹⁸F]FBuEA) and the preparation method for HPLC non-radioactive standards. Its chemical structure is shown in the following:

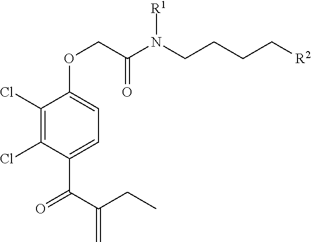

In the precursor, R¹ represents a protective group for the amide functional group; R² represents leaving group; or R¹ represents carboxyl group, R² represents p-tosyloxy, methane sulfonyloxy group or trifluoromethane sulfonyloxy group or bromine (Br). For the HPLC non-radioactive standards, R¹ represents a protective group for the amide functional group and hydrogen, R² represents fluorine.

### BACKGROUND OF THE INVENTION

1. Field of the Invention

The present invention is related to a nuclear medical imaging radiotracer. Especially, it refers to a precursor for a no-carrier-added N-(4-[¹⁸F]fluorobutyl)-Ethacrynic amide([¹⁸F]FBuEA) and the preparation method for non-radioactive high performance liquid chromatography (HPLC) standards.

2. Description of the Prior Art

Presently, the contrast agent for the popular detection equipment for radiopharmaceuticals, like magnetic resonance imaging (MRI), requires high magnetic susceptibility for atoms to undergo magnetic labeling. Metal atoms are not suitable for labeling small drug molecules. No matter it is ultrasonic wave or magnetic resonance imaging (MRI) that is used with the contrast agent, their signal strength is far worse than that by gamma ray, it does not allow the use of a trace amount of drug for detection. Nevertheless, proton emission tomography (PET) or single-photon emission computed tomography (SPECT) makes the analysis of biochemical reactions in vivo possible. The operation principle is based on the use of radioactive tracers, which are compounds that carry radioactive nuclides. Both single-photon emission tomography and proton emission tomography are nuclear medical imaging technologies. The selection of radioactive nuclide must consider “match of nuclide and detection equipment” and “nuclide biocompatibility”. It the nuclide emits gamma ray, SPECT can be used. If the nuclide emits proton, because gamma ray of 511 keV in opposite direction is emitted during proton-electron annihilation, PET can be used. Most biocompatible nuclides emit protons, such as carbon-11, nitrogen-13, oxygen-15, fluorine-18, bromine-75, bromine-76, bromine-80 g, iodine-124. Since the biocompatibility for fluorine compounds is due to the similarity in van der Waals radius with hydrogen, they can replace hydrogen. Studies have found ethacrynic acid has anti-cancer potential. It aims at the protein that is glutathione S-transferaseP1-1 (GSTP1-1). This type of protein occurs in many cancer cells. Some studies also show that it is related to drug resistance in cancer chemotherapy. Recent researchers have found butyl modification for ethacrynic acid to form ethacrynic acid butyl-ester can enhance the ability to kill cancer cells. However, ester bond is not as stable as amide bond. The inventor used amide bond to replace ester bond and expected to generate clinical value. Fluorine-18 butyl ethacrynic amide has the following chemical structure (2):

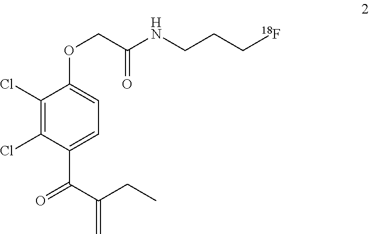

### SUMMARY OF THE INVENTION

The primary objective for the present invention is to provide a precursor for a no-carrier-added N-(4-[¹⁸F]fluorobutyl)-Ethacrynic amide([¹⁸F]FBuEA) and the preparation method for non-radioactive high performance liquid chromatography (HPLC) standards. The [18F]FBuEA precursor (1) has the following chemical structure:

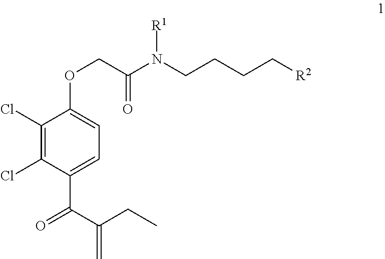

In the synthesis of the precursor for [18F]FBuEA, R1 represents a protective group for the amide functional group and R2 represents a leaving group; or R1 can be carboxyl group and R2 can be p-tosyloxy, methane sulfonyloxy group or trifluoromethane sulfonyloxy group or bromine (Br) group.

In the synthesis of the HPLC non-radioactive standards, R1 represents a protective group for the amide functional group and hydrogen and R2 represents fluorine. N-(4-[18F]fluorobutyl)-Ethacrynic amide injection fluid can be used for tumor diagnostics and curative effect tracking by nuclear medical imaging.

### DETAILED DESCRIPTION OF THE INVENTION

Fluorine-18 labeled precursor, Toluene-4-sulfonic acid 4-(tert-butoxycarbonyl-Ethacrynamino)-butyl ester, (9) and its HPLC non-radioactive standards, {2-[2,3-Dichloro-4-(2-methylene-butyryl)-phenoxy]-acetyl}-(4-fluoro-butyl)-carbamic acid tert-butyl ester (10), and

2-[2,3-Dichloro-4-(2-methylene-butyryl)-phenoxy]-N-(4-fluoro-butyl)-acetamide (11) have the following preferred process embodiments:

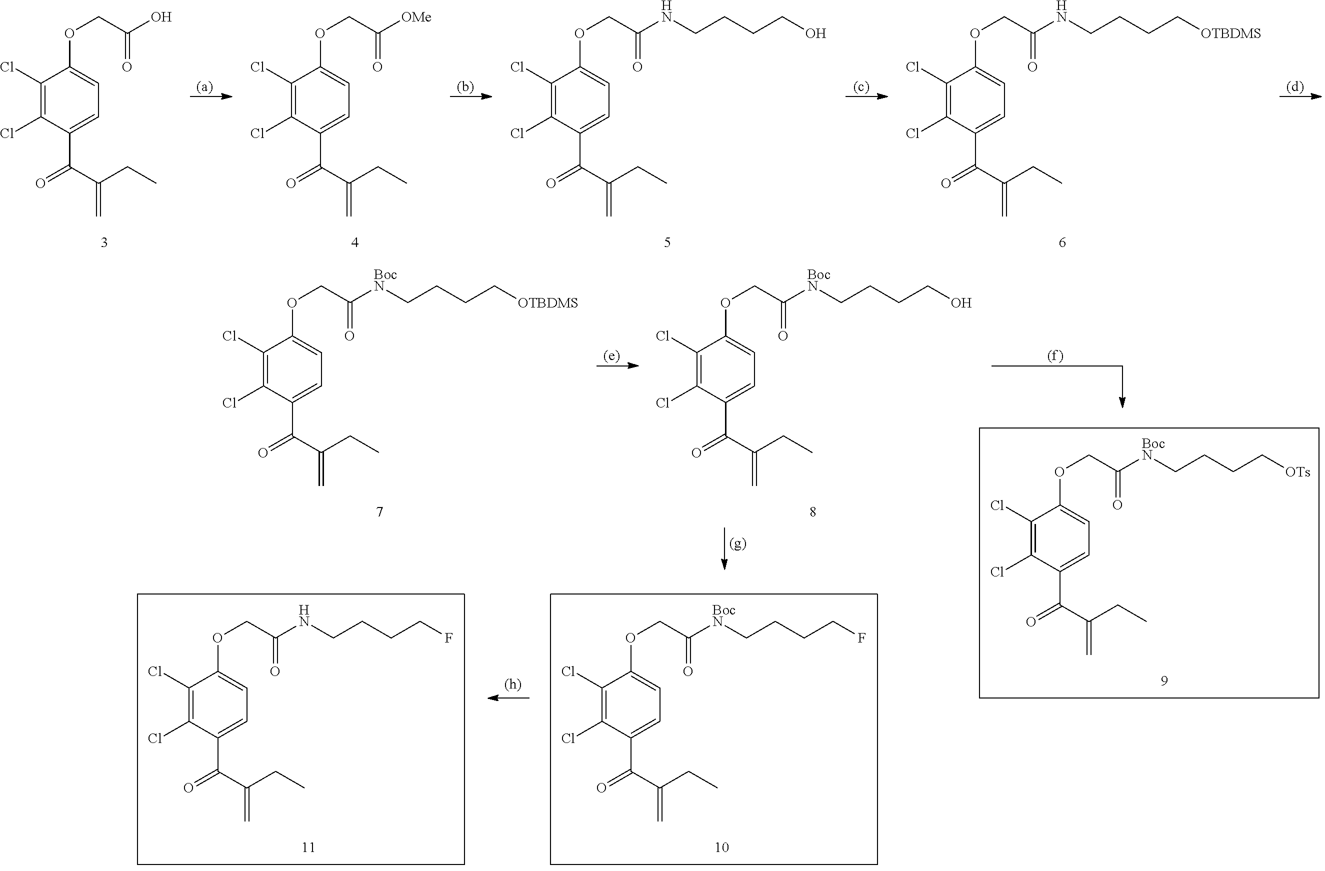

(a) [2,3-Dichloro-4-(2-methylene-butyryl)-phenoxy]-acetic acid methyl ester (methyl ethacrynate) (4) has the following synthesis procedures:

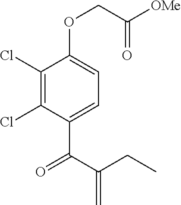

1) Add nitrosomethylurea 1.16 g (1.5 eq, 9.90 mmol) light yellow powder into a round-bottom flask containing 80 mL Et₂O and cover it with nitrogen blanket. Put it under the ice-bath condition.

2) Use 16 mL secondary water to dissolve KOH 1 g (2.7 eq, 17.82 mmol) to make potassium hydroxide solution. Put this KOH solution under ice bath by a round-bottom flask containing nitrosomethylurea. At this moment, nitrosomethylurea gets dissolved to produce diazomethane (CH₂N₂, by ˜−23° C.), a yellow gas (quick consumption to prevent gas loss). Use an ice-bath separating funnel to separate and collect organic layer. Add KOH under ice bath condition for dewatering until dissolution limit.

3) Dissolve the starting material (3), ethacrynic acid 2 g (1 eq, 6.60 mmol), into 20 mL EtOAc. Then, add previously prepared diazomethane (CH₂N₂, by ˜−23° C.) ether solution until no consumption on yellow diazomethane and the occurrence of light yellowness (after TLC shows spreading by EtOAc/n-Hexane (1/4), it simultaneously shows the disappearance of starting material and the formation of products.

4) Use little amount of acetic acid to interrupt excessive diazomethane and make reaction solution clear. Use rotary vacuum concentrator at 40° C. to remove excessive reaction solvents. Last, EtOAc/n-hexane 3/17 is used for chromatographic separation by silicone column. A semi-transparent soft solid (4) approximately 1.9 g (yield: ˜90%) can be obtained.

(b) 2-[2,3-Dichloro-4-(2-methylene-butyryl)-phenoxy]-N-(4-hydroxy-butyl)-acetamide (5) has the following synthesis procedures:

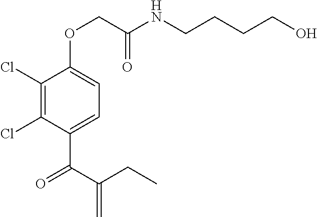

1) Use 40 Ml dry DMF (after dewatering by CaH₂ and reduced-pressure distillation) to dissolve the starting material (4) (1.1 g, 1 eq, 2.94 mmol) and put id nitrogen system.

2) Add about 5 mL triethylamine(Et₃N, after KOH dewatering and reduced-pressure distillation, ˜10 eq, 29.4 mmol, d 0.71). Add 4-amino-1-butanol for about 1.5 mL (˜5.5 eq, 162 mmol, d 0.96). Apply agitation by magnetic stirrer. Put it in oil bath to heat up to 55-60° C. Above the round-bottom flask, a drying tube containing blue silicone gel can prevent water from entering when the system is open to release methanol.

3) After continuous reaction for 8 hours, perform TLC with acetone/n-hexane mixture (1/3). Apply high vacuum to remove solvent. Observe the disappearance of the starting material (4) disappears and the formation of the product (ninhydride, colorless, R_{f}˜0.23).

4) Use vacuum pump and 55° C. rotary vacuum concentrator to remove most reaction solvent. Last, use acetone/n-hexane mixture (1/3→>3/7) for gradient elution on silicone column chromatographic separation to obtain white foamy product (5) for about 255 mg (yield: ˜20%).

(c) N-[4-(tert-Butyl-dimethyl-silanyloxy)-butyl]-2-[2,3-dichloro-4-(2-methylene-butyryl)-phenoxy]-acetamide (6) has the following synthesis procedures:

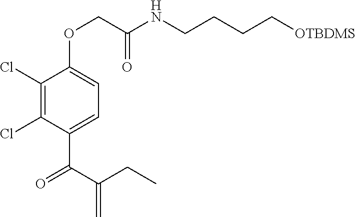

1) Dissolve 220 mg starting material (5) (1 eq, 0.45 mmol) with toluene and apply high-vacuum azeotropic dewatering. Use 11 mL CH₂Cl₂ (from distillation system) for dissolution and put it into a double-neck flask and apply nitrogen.

2) Dissolve TBDMSCl (3.6 eq, 1.62 mmol) and pyridine (0.5 mL, from distillation system) in another double-neck flask with 11 mL CH₂Cl₂ (from distillation system). Then, use syringe to withdraw the mixture into the double-neck flask containing the starting material (5) and connecting to nitrogen supply.

3) Last, add DMAP (dimethylaminopyridine, 1.6 eq, 0.73 mmol, 90 mg) and dissolve it with toluene and perform high-vacuum azeotropic distillation three times for dewatering). Apply agitation with magnetic stirrer and continue reaction at room temperature for about 8 to 9 hours. Perform TLC with acetone/n-hexane mixture 1 and observe the disappearance of starting material (5) and the formation of product (6).

4) Put reactants in a single-neck round-bottom flask. Use rotary vacuum concentrator at 40° C. to remove excessive reaction solvent. Last, use EtOAc/n-hexane (3/7) mixture to perform silicone column chromatographic separation to obtain oily product (6) for about 200 mg (yield: ˜70%).

(d)[4-(tert-Butyl-dimethyl-silanyloxy)-butyl]-{2-[2,3-dichloro-4-(2-methylene-butyryl)-phenoxy]-acetyl}-carbamic acid tert-butyl ester (7) has the following synthesis procedures:

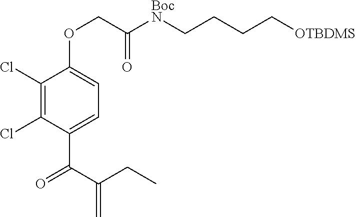

1) Dissolve 200 mg starting material (6) (1 eq, 0.41 mmol) with toluene and apply high-vacuum azeotropic dewatering. Dissolve the mixture with 10 mL CH₂Cl₂ (from distillation system) and put it into a double-neck flask. Apply nitrogen.

2) Withdraw Boc₂O (2.1 eq, 0.2 mL, 0.87 mmol, d 0.95, mp 23° C.) and Et₃N (1.4 eq, 0.08 mL, d 0.73, from reduced-pressure distillation system) with a syringe and put them into the double-neck flask containing the starting material (6) and connecting to nitrogen. Last, add DMAP (dimethylaminopyridine, 1.6 eq, 80 mg, 0.66 mmol) and dissolve the mixture with toluene, and apply high-vacuum azeotropic distillation three times for dewatering). Apply agitation with magnetic stirrer and continue reaction at room temperature for about 12 hours. Perform TLC with EtOAc/n-hexane (3/7) mixture. Observe the disappearance of the starting material (6) and the formation of the product (7).

3) Put the reactant mixtures in a single-neck flask. Use rotary vacuum concentrator at 40° C. to remove the excessive reaction solvent. Last, use EtOAc/n-hexane (1/9) mixture for silicone column chromatographic separation to obtain oily product (7) for about 185 mg (yield: ˜76%).

(e) {2-[2,3-Dichloro-4-(2-methylene-butyryl)-phenoxy]-acetyl}-(4-hydroxy-butyl)-carb amic acid tert-butyl ester (8) has the following synthesis procedures:

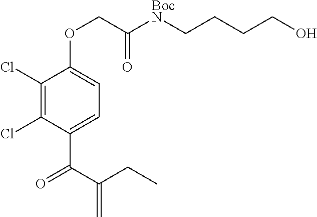

1) Dissolve about 200 mg starting material (7) (1 eq, 0.34 mmol) with about 10 mL THF from distillation system and put in a magnetic stirrer.

2) Mix 1M TBAF/THF (5% water) about 0.34 mL (˜1.0 eq) and acetic acid (AcOH, 1.0 eq, 0.02 mL, d 1.05, 0.33 mmol) and 10 mL THF. Put the mixture into a round-bottom flask containing the starting material and with magnetic stirrer agitation conduct the reaction at room temperature over night (16 hrs). Perform TLC with EtOAc/n-hexane (3/7) mixture. Observe the disappearance of the starting material and the formation of the product (8) (5<R_{f}<6,7).

3) Use rotary vacuum concentrator at 40° C. to remove excessive reaction solvent. Last, use EtOAc/n-hexane (3/7) mixture to perform silicone column chromatographic separation to obtain oily product (8) about 120 mg (yield: ˜75%).

(f) Toluene-4-sulfonic acid 4-(tert-butoxycarbonyl-{2-[2,3-dichloro-4-(2-methylene-butyryl)-phenoxy]-acetyl}-amino)-butyl ester (9) (precursor) has the following synthesis procedures:

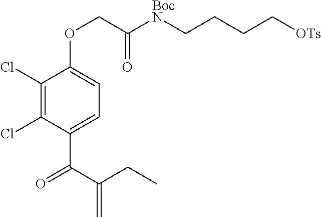

1) Dissolve 30 mg starting material (8) (1 eq, 0.063 mmol) with toluene and apply high-vacuum azeotropic distillation. Add in magnetic stirrer. Connect to nitrogen system. Dissolve the mixture with 2 mL dichloromethane (from distillation system).

2) Put the round-bottom reaction flask containing the starting material under ice bath condition (0-5° C.). Add TsC (1.25 eq, 15 mg, 0.079 mmol, co-crystal from EtOAc/n-hexane mixture) that is pre-dissolved in dichloromethane and add 0.08 mL pyridine (from distillation system).

3) After reaction under ice bath condition for half hour, put the reaction flask (containing nitrogen balloon) in a refrigerator (4° C.) over night. Perform TLC with EtOAc/n-hexane=1. There will still be formation of some product (9). Although the starting material (8) decreases, it does not completely disappear (add a little TsCl (0.5 eq) or leave it for another day, there is a little improvement, but not significant).

4) After rotary vacuum concentrator, use EtOAc/n-hexane (1/4) mixture to perform silicone column chromatographic separation on the crude product to obtain transparent oily product (9) for about 5-6 mg (yield: ˜20%) and recycle to obtain the starting material (8) for about 10 mg (yield:˜33%).

(g) {2-[2,3-Dichloro-4-(2-methylene-butyryl)-phenoxy]-acetyl}-(4-fluoro-butyl)-carbamic acid tert-butyl ester (10): (HPLC nonradioactive standards)

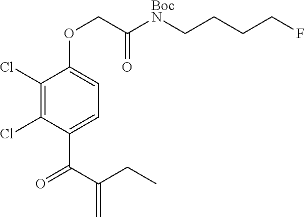

1) Dissolve 50 mg starting material (8) (1 eq, 0.106 mmol) with toluene and apply azeotropic dewatering. Use about 5 mL dichloromethane (from distillation system) to dissolve the mixture and transfer it to a double-neck flask containing magnetic stirrer and under nitrogen system.

2) Put the double-neck flask containing the starting material in a −78° C. low-temperature reactor [symbol] . Gradually add DAST (diethylaminosulfur trifluoride) (˜1.5 eq, 20 uL, 0.15 mmol, d 1.22, by 30-32° C.) and at −78° C. apply magnetic stirrer agitation for 30 min-1 h. Then, the double-neck flask slowly returns to room temperature. Perform TLC with EtOAc/n-hexane (2/3 & 3/7) mixture. Observe the disappearance of the starting material (8) and the formation of the product (10).

3) At TLC original point (R_{f}=0) there is formation of some unknown materials. Add 5 mL cold saturated NaHCO_{3(aq)} and then use dichloromethane for extraction three times (3×10 mL). Collect the organic layer and use Na₂SO₄₍ₛ₎ for dewatering. Use gravity filtration to remove Na₂SO₄₍ₛ₎. Use reduced-pressure concentrator to withdraw dichloromethane. Last, use EtOAc/n-hexane (1/4) mixture to perform silicone column chromatographic separation to obtain transparent oily product (10) for about 22 mg (yield: ˜45%).

(h) 2-[2,3-Dichloro-4-(2-methylene-butyryl)-phenoxy]-N-(4-fluoro-butyl)-acetamide (11): (HPLC nonradioactive standards)

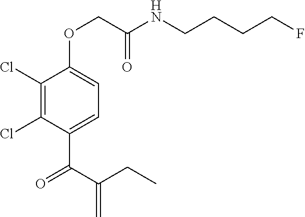

1) Dissolve 15 mg starting material (10) with dichloromethane. Add a magnetic stirrer and 0.25 mL TFA. Apply agitation and conduct reaction at room temperature for half hour. Perform TLC with EtOAc/n-hexane mixture 1. Observe the disappearance of the starting material (10) and the formation of the product (11).

2) Start rotary vacuum concentrator at 40° C. Last, use EtOAc/n-hexane (3/7) mixture to perform silicone column chromatographic separation to obtain white solid product (11) for about 8 mg (yield: ˜70%, mp 94-96° C.).

What is claimed is:

1. A precursor for a N-(4-[¹⁸F]fluorobutyl)-Ethacrynic amide([¹⁸F]FBuEA) and the preparation method for non-radioactive high performance liquid chromatography (HPLC) standards, the chemical structure is shown in the following: 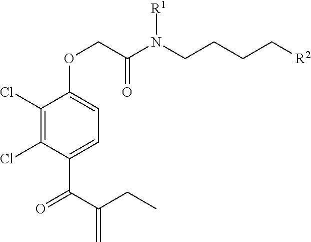
which can be made into N-(4-[¹⁸F]fluorobutyl)-Ethacrynic amide as fluorine-18 labeled precursor and HPLC non-radioactive standards;
for the precursor,
R¹: represents the protective group for amide group, which is carboxyl group;
R²: represents the leaving group, which is p-tosyloxy, methane sulfonyloxy group or trifluoromethane sulfonyloxy group or bromine (Br) group;
for HPLC non-radioactive standards,
R¹: represents protective group for acid group or no protective group, which is carboxyl group;
R²: represents fluorine (F).

2. The precursor of the claim 1, wherein the compounds prepared for the production of the precursor include:
2-[2,3-Dichloro-4-(2-methylene-butyryl)-phenoxy]-N-(4-hydroxy-butyl)-acetamide, N-[4-(tert-Butyl-dimethyl-silanyloxy)-butyl]-2-[2,3-dichloro-4-(2-methylene-butyryl)-phen oxy]-acetamide,
[4-(tert-Butyl-dimethyl-silanyloxy)-butyl]-{2-[2,3-dichloro-4-(2-methylene-butyryl)-phenox y]-acetyl}-carbamic acid tert-butyl ester,
{2-[2,3-Dichloro-4-(2-methylene-butyryl)-phenoxy]-acetyl}1-(4-hydroxy-butyl)-carbamic acid tert-butyl ester.

3. The compounds of the claim 2, wherein the compound {2-[2,3-Dichloro-4-(2-methylene-butyryl)-phenoxy]-acetyl}1-(4-hydroxy-butyl)-carbamic acid tert-butyl ester is prepared for the production of Toluene-4-sulfonic acid 4-(tert-butoxycarbonyl-{2-[2,3-dichloro-4-(2-methylene-butyryl)-phenoxy]-acetyl}1-amino)-butyl ester.

4. The compounds of the claim 2, wherein the compound {2-[2,3-Dichloro-4-(2-methylene-butyryl)-phenoxy]-acetyl}1-(4-hydroxy-butyl)-carbamic acid tert-butyl ester is prepared for the production of {2-[2,3-Dichloro-4-(2-methylene-butyryl)-phenoxy]-acetyl}1-(4-fluoro-butyl)-carbamic acid tert-butyl ester.

5. The compounds of the claim 4, wherein the compound {2-[2,3-Dichloro-4-(2-methylene-butyryl)-phenoxy]-acetyl}1-(4-fluoro-butyl)-carbamic acid tert-butyl ester is prepared for the production of 2-[2,3-Dichloro-4-(2-methylene-butyryl)-phenoxy]-N-(4-fluoro-butyl)-acetamide.

6. A N-(4-[¹⁸F]fluorobutyl)-Ethacrynic amide([¹⁸F]FBuEA) injection which is made from the Toluene-4-sulfonic acid 4-(tert-butoxycarbonyl-{2-[2,3-dichloro-4-(2-methylene-butyryl)-phenoxy]-acetyl}1-amino)-butyl ester can be used for tumor diagnostics and curative effect tracking by nuclear medical imaging.
